(19) United States
(12) Patent Application Publication
(10) Pub. No.: US 2009/0030215 A1
(43) Pub. Date: Jan. 29, 2009

(54) METHOD FOR PRODUCTION OF 5-HYDROXYMETHYL-2-FURFURAL FROM FRUCTOSE

(76) Inventors: Christine DIGNAN, Decatur, IL (US); Alexandra J. SANBORN, Lincoln, IL (US)

Correspondence Address: MCDERMOTT WILL & EMERY LLP, 600 13TH STREET, N.W., WASHINGTON, DC, 20005-3096, US

(21) Appl. No.: 12/176,028

(22) Filed: Jul. 18, 2008

Related U.S. Application Data
(60) Provisional application No. 60/950,459, filed on Jul. 18, 2007.

(51) Int. Cl. C07D 307/50

(52) U.S. Cl. 549/488

(57) ABSTRACT

A method of producing HMF by mixing or agitating an aqueous solution of fructose and inorganic acid catalyst with a water immiscible organic solvent to form an emulsion of the aqueous and organic phases. The mixture is heated in a flow-through reactor at elevated pressures and then separated into aqueous and organic phases to obtain HMF. The aqueous phase and the organic phase are mixed with an in-line mixer prior to, preferably, immediately before addition of the biphasic reaction mixture into the reactor. After separation, HMF is recovered from the aqueous and organic phases to obtain high yields of HMF without the presence of insoluble solid impurities.

### CROSS REFERENCE TO PROVISIONAL APPLICATION

This application is based upon and claims the benefit of priority from Provisional U.S. Patent Application 60/950,459 (Attorney docket No. 010253-0014) filed on Jul. 18, 2007, the entire contents of which are incorporated by reference herein.

### BACKGROUND

1. Technical Field

This disclosure relates to the production of 5-hydroxymethyl-2-furfural (HMF). More particularly, the invention relates to the production of 5-hydroxymethyl-2-furfural using acid catalyzed dehydration of fructose at elevated temperatures in a bi-phasic system.

2. Introduction

A major product in the acid-catalyzed dehydration of fructose is 2-hydroxymethyl-5-furfuraldehyde, also known as hydroxymethylfurfural which is abbreviated, HMF. The structure of HMF is shown below:

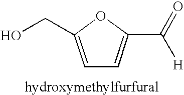

While not being bound by theory, it is generally believed that fructose is converted to HMF via an acyclic pathway, although evidence also exists for the conversion to HMF via cyclic fructofuransyl intermediate pathways. Regardless of how the mechanism of HMF formation occurs, the intermediate species formed during the reaction may in turn undergo further reactions such as condensation, rehydration, reversion and other rearrangements, resulting in a plethora of unwanted side products. Below is one proposed pathway for the conversion of fructose to HMF:

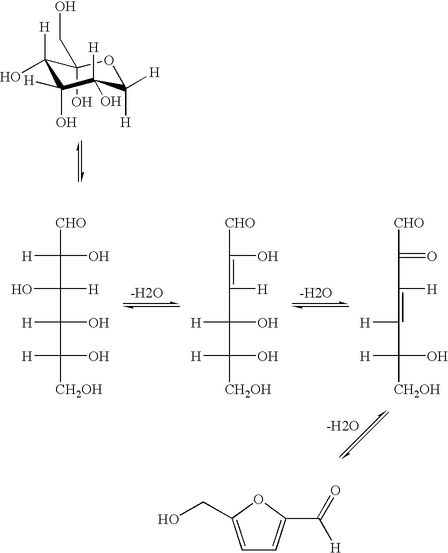

HMF has been reported to have antibacterial and anticorrosive properties. HMF is also a key component, as either a starting material or intermediate, in the synthesis of a wide variety of compounds, such as furfuryl dialcohols, dialdehydes, esters, ethers, halides and carboxylic acids. Examples of carboxylic acids that can be derived from HMF are levulinic acid and formic acid. One important reaction of HMF is the organic oxidation to 2, 5-furandicarboxylic acid, a compound that has been suggested for use as a monomer in the production of plastics. In addition, HMF has great potential as a biofuel, which are fuels derived from biomass and are considered promising alternatives to fossil fuels. HMF is also currently under investigation as a treatment for sickle cell anemia. In short, HMF is an important chemical compound and a method of synthesis on a large scale to produce HMF absent significant amounts of impurities, side products and remaining starting material has been sought for nearly a century.

Agricultural raw materials such as starch, cellulose, sucrose or inulin are inexpensive starting materials for the manufacture of hexoses, such as glucose and fructose. As shown above, these hexoses can in turn, be converted to HMF. The dehydration of sugars to produce HMF is well known. HMF was initially prepared in 1895 from levulose by Dull (Chem. Ztg., 19, 216) and from sucrose by Kiermayer (Chem. Ztg., 19, 1003). However, these initial syntheses were not practical methods for producing HMF due to low conversion of the starting material to product.

British Patent No. 600,871 describes an improved method of manufacturing HMF in which a solution of a carbohydrate is heated under pressure at a temperature from 130° to 230° C. depending upon the pH of the reaction and nature of the carbohydrate material. The reaction is generally carried out in an autoclave under a hydrogen atmosphere.

U.S. Pat. No. 2,929,823 describes producing HMF from sugars at temperatures from 250° to 380° C. using very short reaction times, on the order of 0.1 to 180 seconds. The reaction is carried out by quickly heating an aqueous solution of sugar, glucose, fructosans, fructose, sucrose, hydrolyzed wood or starch. The reaction may be performed either by injecting super-heated steam into the solution or by passing the solution through a set of reactor coils of very small diameter in the presence of an extraction solvent, such as furfural.

However, with the two above described methods, yields are low, on the range of 40% or less, and side products, such as tarry or solid materials are formed. The side products must be removed and may interfere with the purification of the HMF. For example, one such side material, humin, is a brown to black, fluffy solid which is almost completely insoluble in water, base, acids and organic solvents of all types. It coats the sides of reaction vessels and serves as an efficient thermal insulator, thereby causing poor heat transfer. Humin also induces emulsification of the aqueous phase with various extraction solvents and complicates the recovery of HMF.

Another drawback of the above described processes is that they are inefficient. A large amount of the starting material remains unconverted to product, requiring difficult separation of the reaction mixture and increased material cost per unit of HMF produced.

U.S. Pat. No. 2,750,394 describes a method of manufacturing HMF using a mixture of an aqueous phase containing levulose, sucrose or black strap molasses and an organic phase containing low molecular weight alcohols at temperatures from about 125° to 225° C. in sealed glass tubes or an autoclave. These methods require long reaction times and multiple reaction steps in order to obtain the product.

One recent method for the production of HMF from fructose involves the conversion of aqueous fructose solutions of from 10 to 50 wt % in a two-phase reactor system in which the aqueous fructose solution is combined with either DMSO or PVP and then continuously extracted into an organic phase. However, the high boiling DMSO and PVP requires expensive and time consuming vacuum evaporation or vacuum distillation steps for removal, as well as large amounts of organic solvent for extracting the HMF from the aqueous phase.

In summary, the synthesis of HMF from fructose, is a complex reaction which, depending upon the temperature, pressure, solvent, duration and other reaction conditions, can result in a wide variety of side products and has historically resulted in low conversion rates of starting material to product. There is a need therefore, for a method of producing HMF that eliminates or reduces the above mentioned problems.

### SUMMARY

In order to overcome the above mentioned problems, the disclosure provides a method of producing HMF by mixing or agitating an aqueous solution of fructose and inorganic acid catalyst with a water immiscible organic solvent. The mixture is then heated in a reactor at elevated pressures and then separated into aqueous and organic phases to obtain HMF. Preferably, the aqueous phase and the organic phase are mixed with an in-line mixer prior to, preferably, immediately before addition of the biphasic reaction mixture into the reactor.

In one embodiment, there is provided a method for manufacturing hydroxymethylfurfural comprising the steps of:

a) mixing a first aqueous phase comprising fructose, a first organic phase comprising at least one low molecular weight alcohol, and an inorganic acid to form a biphasic mixture;

b) passing the mixture through a heated continuous flow reactor;

c) separating the biphasic mixture into a second aqueous phase and a second organic phase; and

d) removing hydroxymethylfurfural from the second aqueous phase and the second organic phase. Preferably, the first aqueous phase and the first organic phase are mixed with an in-line mixer.

Another embodiment provides a method for manufacturing hydroxymethylfurfural described above, wherein the concentration of fructose in the first aqueous phase is from 25 to 70 wt/vol %.

In a preferred embodiment of the method, the inorganic acid comprises hydrochloric acid or sulfuric acid. In another embodiment, the pH of the bi-phasic mixture due to addition of the inorganic acid is from about 1.5 to 3.5, and preferably from 1.5 to 2.5.

In another embodiment, the first organic phase comprises an organic solvent selected from the group consisting of a fusel oil, a pentanol and a butanol.

Preferably, the ratio of volumes of the first aqueous phase to the first organic phase is from 1:0.25 to 1:4. The mixing of the first aqueous phase and the first organic phase preferably creates a biphasic emulsion.

In another preferred embodiment, the mixture is heated to a temperature of from about 200° C. to about 300° C., preferably from about 240° C. to about 270° C. in the continuous flow reactor during step b).

In yet another embodiment, the mixture formed in step a) is passed through the continuous flow reactor during step b) at a pressure greater than 150 psig and less than 1200 psig, preferably at a flow rate of between about 2 and about 6 ml/minute.

In another embodiment, at least 59% of the HMF partitions into the organic phase.

In a further preferred embodiment, the hydroxymethylfurfural in the second aqueous phase is purified by passing the second aqueous phase through an ion-exchange resin.

Two advantages of the method as described herein are the obtention of a high rate of conversion of fructose into HMF, and a reduction in the formation of side products observed with prior art methods. Another advantage is the reduction in the amount of solvent used, for both the aqueous phase and organic phase, in relation to the amount of fructose. This is accomplished by utilizing high fructose concentrations in the initial aqueous phase. This results in lower cost in materials, reduced time for removal of solvent and a reduced negative impact on the environment as a result of the smaller amount of solvents used.

### DETAILED DESCRIPTION

The present disclosure provides a method for manufacturing hydroxymethylfurfural comprising the steps of mixing a first aqueous phase comprising fructose and an inorganic acid with a first organic phase comprising at least one organic solvent. This mixture is passed through a heated continuous flow reactor and then separated into a second aqueous phase and a second organic phase. Hydroxymethylfurfural is removed from the second aqueous phase and the second organic phase in high yield.

The fructose in the aqueous phase may be in any form, such as a concentrated syrup, a particulate or crystalline solid, and may be obtained from any commercially available source. The fructose may be formulated in the aqueous phase at a concentration from about 15 to about 70 wt/vol %, preferably from about 20 to about 60 wt/vol %, and most preferably from about 25 to about 45 wt/vol %.

The aqueous phase used in the method may also comprise a catalytic amount of inorganic acid. Generally, any acid known in the art that effectively lowers the pH of the reaction mixture to afford hydrolysis of fructose, while reducing the occurrence of competing side reactions may be used. Examples of inorganic acids used in present disclosure include, but are not limited to hydrochloric acid (HCl) and sulfuric acid (H₂SO₄). Preferably, the pH of the inorganic acid in the aqueous phase is about 1.0 to 4.0, more preferably from about 1.5 to 3.5 and most preferably from about 1.5 to 2.5.

The organic phase used in the method preferably is an organic solvent or combination of organic solvents capable of forming a bi-phasic mixture with aqueous solutions. In addition, the organic phase preferably is capable of solubilizing HMF at room temperatures (generally about 25° C.) or higher temperature. In a preferred embodiment, the organic phase comprises at least one low molecular weight alcohol. Many low molecular weight alcohols are capable of both forming a bi-phasic mixture with aqueous solutions and solubilizing HMF. Examples of low molecular weight alcohols utilized in the present disclosure are fusel oil, isoamyl alcohol, butanol, isopentyl alcohol and similarly related compounds. Fusel oil is a by-product of carbohydrate fermentations whose main components are isopentyl alcohol and 2-methyl-1-butanol, and to a lesser degree contains isobutyl alcohol, n-propyl alcohol, and small amounts of other alcohols, esters and aldehydes. One advantage of utilizing low molecular weight alcohols is their ability to abstract the HMF produced in the reaction into the organic layer, allowing the reaction equilibrium to shift towards the final reaction products. Lower molecular weight alcohols are easily recovered by evaporation and recycled. Solvent loss is eliminated making this process very efficient.

The aqueous phase and the organic phase are generally mixed to form a bi-phasic mixture before the reactants are added to the reactor. A variety of known means in the art to mix solutions may be employed. For example, the aqueous phase and organic phase may be combined in a single vessel prior to pumping the mixture into the reactor, or mixing may be achieved by the use of an in-line mixer. Use of an in-line mixer ideally results in the formation of a fine emulsion of the aqueous and organic phases. Another advantage to using an in-line mixture is that the bi-phasic mixture is less likely to separate into aqueous and organic phases during the reaction. One type of in-line mixer that is used in one embodiment of the present disclosure is the Analytical Scientific Instruments SS 500 μL in-line mixer. Two types of high-pressure pumps used are the Eldex high pressure pump model 1HM and the Hitachi L-6000 pump.

The ratio of the volume of the aqueous phase to the volume of the organic phase used in the present method generally ranges from about 1:0.1 to about 1:8 (aqueous phase:organic phase, or “aq:org”), and preferably from about 1:0.25 to about 1:4. One important advantage to utilizing a low amount of organic phase is the lowered cost of removing the solvent after the completion of the reaction. It is desirable to reduce the amount of organic solvent in the reaction to ease the burden on the environment and lower the time and energy necessary to remove it. However, the use of too little organic solvent may result in a lower yield of HMF due to a diminished ability for the HMF to pass into the organic phase.

After forming the biphasic mixture by mixing the aqueous and organic phases, the mixture is then pumped into a thermal flow-through reactor. The present method for producing HMF is a continuous process as opposed to a batch reaction method, and therefore utilizes a flow-through reactor. A flow-through reactor is a device that allows chemical reactions to be performed as a continual process in which reactants are continually added to the input end of the reactor and product is continually collected from the output end. A flow-through reactor provides the user good control over reaction conditions, such as heat transfer, time and mixing. Other terms of art synonymous with flow-through reactor are tube reactor and continuous flow reactor.

The heat source applied to the flow-through reactor may be any type of heating source known to those skilled in the art that provides constant and uniform heat and is capable of heating the coil to the temperature at which the reaction is conducted. Examples of a heating source used in some embodiments of the present disclosure are a heated fluidized sand bath or a hot oil bath.

Preferably, the reaction tube used in the present method has a smaller diameter, which provides a uniform temperature can be maintained throughout the entire reaction. As such, problems associated with large scale batch reactors such as localized temperature gradients and uneven mixing of reactants may be eliminated. Furthermore, as the flow rate may be controlled via the pressure pump, the reaction time can be easily controlled by utilizing information on the flow rate and the reactor volume. For example, to increase the reaction time, a skilled artisan would know to decrease the flow rate or use a larger diameter coil.

Any flow rate that results in the conversion of fructose to HMF without significant negative reactions of the HMF may be used in the present method. Flow rates that are too fast do not allow enough time for the fructose to be completely converted into HMF. Conversely, flow rates that are too slow result in an increased reaction times, and the possible formation of side products and decomposition of HMF. In a preferred embodiment of the present invention, a thermal flow-through reactor having a 1/16″ OD and having a 36″ length coil, a flow rate of from about 3 ml/min to about 6 ml/min is used, resulting in an effective conversion of fructose to HMF. However, it is well known to those skilled in the art that variables such as pressure and concentration of fructose in the mixture will result in the need for minor experimental testing to achieve the optimum yields of HMF. Preferred flow rates are from about 1 to about 10 ml/min. More preferably, the flow rate is from about 2 to about 7 ml/min. Most preferably, the flow rate is from about 4 to about 5 ml/min. In addition, the flow rate of the biphasic emulsion is related to the residence time of the mixture in the flow-through reactor. The residence time, and accordingly, the reaction time of the reaction may be converted from the flow rate if the ID and length of the coil is known. For example, a flow rate of 4 ml/min in a 36″ coil having an inner diameter of 1/20″ is 0.29 min, or 17.4 seconds. Accordingly, preferable reaction times range from about 4 to about 60 seconds. More preferably, the reaction times range from about 10 to about 45 seconds. Most preferably, the reaction times range from about 15 to about 20 seconds.

The reaction conditions for the conversion of fructose to HMF in the present method include elevated temperatures and pressures. The temperature for the reaction may be from about 240° to 270° C. The pressures that are used in the method of the present disclosure are generally from about 150 psig to 1200 psig.

After the reaction mixture passes through the reactor, the mixture separates back into a second aqueous phase and a second organic phase. HMF produced during the reaction is present in both phases in roughly equal amounts. However, unreacted fructose is generally found in the aqueous phase. One great advantage of this process is that substantially no solid impurities are formed, which allows for a simpler purification of the final product. As used herein, substantially no formation of solid impurities means the solid impurities are below visual detection with the unaided eye. Furthermore, those skilled in the art would readily understand the meaning of substantially no formation of solid impurities.

After separation of the bi-phasic mixture into a second aqueous and second organic phase, the HMF may be separated from the solvent by various known methods in the art. In one embodiment of the present invention, HMF is removed from the second aqueous phase by passing the second aqueous phase through an ion-exchange resin, such as Lewatit S7768. Other methods to remove the HMF from the organic phase are vacuum evaporation and vacuum distillation, although any process known in the art may be used.

### EXAMPLES

### Example 1

30 to 36 grams of crystalline fructose was dissolved in deionized water to make an 0.1 L (30% or 36% wt/vol) aqueous fructose solution. The fructose solution was combined with an amount of inorganic acid and an amount of fusel oil in an Analytical Scientific Instruments SS 500 μL in-line mixer as shown in Table 1. Five solutions were prepared in this manner. Each solution was pumped separately under pressure through a 1/16″ OD, 1/20″ ID stainless steel tube reactor 36″ in length, heated by an oil bath at various temperatures, pressures and flow rates which are listed in Table 1. The flow-through reactor is designed with a high pressure pump which feeds the biphasic mixture through a 1/16″ outer diameter and 1/20″ or 1/33″ inner diameter stainless steel tube connected at a zero dead volume (ZDV) union to a coiled section of 1/16″ outer diameter and 1/20″ or 1/33″ inner diameter stainless steel tubing submerged in a heat source. This coiled section of tubing then communicates with a second ZDV union to a 12″ section of similar tubing, which is connected to a pressure regulating valve further connected to an outlet for the reaction mixture to leave the reactor.

TABLE 1

| | Amt | | | | | Inorganic |
|---|---|---|---|---|---|---|
| Run # | Fructose | Flow rate | Pressure | aq:org | Temp | acid |
| (LN #) | (% in aq) | (ml/min) | (psig) | ratio | (° C.) | (wt %) |
| 1 | 33 | 4 | 1012 | 1:3 | 263 | 0.2 H₂SO₄ |
| 2 | 30 | 5 | 1056 | 1:4 | 270 | 0.2 H₂SO₄ |
| 3 | 30 | 4 | 995 | 1:3 | 270 | 0.2 H₂SO₄ |
| 4 | 30 | 4 | 1018 | 1:1 | 270 | 0.2 H₂SO₄ |
| 5 | 36 | 4 | 818 | 4:1 | 263 | 0.2 H₂SO₄ |

After the reaction, the bi-phasic mixture separated into a second aqueous phase and second organic phase. The second aqueous phase and second organic phase were analyzed after the reaction using Shimadzu 10 liquid chromatography mass spectrograph. The results of Experiment 1 are shown in Table 2 below:

TABLE 2

| | | | | | | Total |
|---|---|---|---|---|---|---|
| | | | | Formic | Levulinic | combined % |
| | | Fructose | HMF | acid | acid | molar yield |
| Run # | Phase | (g/kg) | (g/kg) | (g/kg) | (g/kg) | HMF |
| 1 | Aq | 1.01 | 54.10 | 3.54 | 3.66 | 105.1 |
| | Org | 0 | 83.17 | 0 | 0.80 | |
| 2 | Aq | 15.48 | 23.88 | 1.45 | 0.60 | 65.4 |
| | Org | 0 | 38.71 | 0.31 | 0.35 | |
| 3 | Aq | 6.92 | 29.55 | 2.08 | 1.50 | 60.4 |
| | Org | 0 | 45.14 | 0 | 0.41 | |
| 4 | Aq | 0.38 | 56.23 | 4.09 | 4.09 | 60.1 |
| | Org | 0 | 101.82 | 0 | 0.83 | |
| 5 | Aq | 61.74 | 79.59 | 0 | 1.65 | 81.1 |
| | Org | 2.4 | 125.09 | 0 | 0 | |

As can be seen in Table 2, a high yield of HMF can be obtained through the method described in the present disclosure. The method also converts a high amount of fructose into product, leaving a low amount of fructose unreacted, as compared to other known processes in the prior art. Furthermore, the amount of the side products formic acid and levulinic acid formed during the method is negligible. Another advantageous result is the reduction of the formation of solid impurities and humins that are usually found in the processes of currently known processes.

What is claimed is:

1. A method for synthesis of HMF comprising the steps of:
combining fructose with an acid catalyst in a solvent mixture comprising an aqueous portion and a water immiscible organic solvent portion to form a first biphasic mixture;
agitating the biphasic mixture to form an emulsion;
heating the emulsion in a reactor at a temperature, under a pressure, and for a time sufficient to convert at least 60% of the fructose to HMF and with substantially no formation of solid impurities;
separating the emulsion into a second biphasic mixture comprising an aqueous phase and an organic phase where at least 50% of the HMF partitions into the organic phase and recovering the HMF from at least one of the organic phase and the aqueous phase.

2. The method of claim 1, wherein the water immiscible organic solvent comprises at least one alcohol component selected from the group consisting of: a fusel oil, a pentanol, and a butanol.

3. The method of claim 1, wherein the water immiscible organic solvent is a fusel oil.

4. The method of claim 1, wherein the emulsion is heated to a temperature of 240° C. to 270° C.

5. The method of claim 1, wherein the pressure is between 150 and 1200 psig.

6. The method of claim 1, wherein the pressure is between 400 and 1200 psig.

7. The method of claim 1, wherein the time sufficient to convert at least 60% of the fructose to HMF is from 4 to 60 seconds.

8. The method of claim 1, wherein a molar yield of HMF from fructose is at least 80%

9. The method of claim 1, wherein a ratio of the aqueous portion to the organic solvent portion is between 1:0.1 and 1:8.

10. The method of claim 1, wherein a ratio of the aqueous portion to the organic solvent portion is between 1:0.25 and 1:4.

11. The method of claim 1, wherein a ratio of the aqueous portion to the organic solvent portion is about 1:0.25.

12. The method of claim 1, wherein the reactor is a flow-through reactor in which the heating occurs, and the reaction is conducted continuously by loading the emulsion containing fructose and catalyst at a first section of the reactor and removing the emulsion containing the HMF from a second section of the reactor after heating.

13. The method of claim 1, wherein the HMF is recovered from both the organic phase and the aqueous phase of the second bi-phasic mixture.

14. A method for synthesis of HMF comprising the steps of:
combining fructose with an acid catalyst in a solvent mixture comprising an aqueous portion and a water-immiscible organic solvent portion to form a first biphasic mixture;
agitating the biphasic mixture to form an emulsion;
passing the emulsion under pressure through a heated flow-through reactor for a time sufficient to convert at least 60% of the fructose to HMF and with substantially no formation of solid impurities;
separating the emulsion into a second biphasic mixture comprising an aqueous phase and an organic phase where at least 50% of the HMF partitions into the organic phase; and
recovering the HMF from at least one of the organic phase and the aqueous phase.

15. The method of claim 14, wherein the water immiscible organic solvent comprises at least one alcohol component selected from the group consisting of: a fusel oil, a pentanol, and a butanol.

16. The method of claim 14, wherein the time sufficient to convert at least 60% of the fructose to HMF is from 5 to 60 seconds.

17. The method of claim 14, wherein the emulsion is heated to a temperature of 240° C. to 270° C.

18. The method of claim 14, wherein the pressure is between 150 and 1200 psig.

19. The method of claim 14, wherein a ratio of the aqueous portion to the organic solvent portion is between 1:0.25 and 1:4.

20. A method for synthesis of HMF comprising the steps of:
combining fructose with an acid catalyst in a solvent mixture comprising an aqueous portion and a water-immiscible alcohol solvent portion at an aqueous solvent:organic solvent ratio of 1:0.25 to 1:4 to form a first biphasic mixture;
agitating the biphasic mixture to form an emulsion;
continuously loading an input port of flow-through reactor with the emulsion;
heating the emulsion in the flow through reactor at a temperature of 240° C. to 270° C., under a pressure of 400 to 1100 psig, for a time sufficient to convert at least 80% of the fructose to HMF and with substantially no formation of solid impurities;
continuously removing the emulsion from an output port of the flow-through reactor;
separating the removed emulsion into a second biphasic mixture comprising an aqueous phase and an organic phase where at least 50% of the HMF partitions into the organic phase and
recovering the HMF from at least one of the organic phase and the aqueous phase.
